(19) United States
(12) Patent Application Publication
(10) Pub. No.: US 2020/0367795 A1
(43) Pub. Date: Nov. 26, 2020

(54) SYSTEM AND METHOD FOR BLOOD GLUCOSE MONITORING USING MAGNETIC RESONANCE SPECTROSCOPY

(71) Applicant: New York University, New York, NY (US)

(72) Inventors: Yongxian QIAN, Forest Hills, NY (US); Bei ZHANG, Oakland Gardens, NY (US)

(21) Appl. No.: 16/960,530

(22) PCT Filed: Jan. 17, 2019
(86) PCT No.: PCT/US19/13989

§ 371 Date: Jul. 7, 2020

Related U.S. Application Data
(60) Provisional application No. 62/618,974, filed on Jan. 18, 2018.

(51) Int. Cl. A61B 5/145; A61B 5/05; A61B 5/00; G01R 33/38; G01R 33/465

(52) U.S. Cl. CPC A61B 5/14532; A61B 5/05; G01R 33/465; G01R 33/3808; A61B 5/681

(57) ABSTRACT

A wearable device and method for noninvasive and continuous monitoring of blood glucose level in a patient. The device may include a unilateral permanent magnet generating a static magnetic field to a target area under the skin of the patient, the target area comprising blood vessels and tissue surrounding the blood vessels. The device may also include a transmitter delivering a radiofrequency (RF) field to the target area to excite proton nuclear spins in the target area, wherein at least a portion of the transmitter is positioned between the magnet and the skin. The device may further include a sensor detecting a RF signal from the excited proton nuclear spins in the target area. A processing arrangement may receive data corresponding to the detected RF signal from the sensor and determine a level of blood glucose in the patient based on the data.

### PRIORITY CLAIM

The present application is a National Phase Application of PCT Patent Application Serial No. PCT/US2019/013989 filed on Jan. 17, 2019; which claims priority to U.S. Provisional Application Ser. No. 62/618,974 entitled “Micro Magnetic Resonance Spectroscopy Blood Glucose Monitor” filed on Jan. 18, 2018, the entire contents of the above applications are hereby incorporated by reference herein.

### BACKGROUND

High blood glucose level is a characteristic of diabetes, such as 100-126 mg/dl (or 5.5-7.0 mmol/L) is considered as pre-diabetes and >126 mg/dl (or >7.0 mmol/L) as diabetes, according to the guidelines of American Diabetes Association. Diabetes is a group of metabolic diseases causing both acute (ketoacidosis, nonketotic hyperosmolar coma, and death) and serious chronic complications (heart disease, stroke, kidney failure, foot ulcers, and eye damages). There are three main types of diabetes: Type 1 (due to absolute insulin deficiency), Type 2 (due to insulin resistance and progressive loss of insulin secretion), and Gestational diabetes (due to pregnancy). There were 422 million people with diabetes worldwide in 2014; 90% of them were Type 2 diabetes and annual direct costs were US$827 billion worldwide and $105 billion (22.4 million diabetic people) in the United States. Diabetes is a chronic disease and there is no known cure for it.

Current management of diabetes focuses on keeping both short-term and long-term blood glucose levels within acceptable range as close to normal. This can usually be accomplished with a healthy diet, exercise, weight loss, and medications. Therefore, monitoring glucose levels is a critical part in the management of diabetes and pre-diabetes. Existing devices for performing daily glucose monitoring at home are invasive and painful for the users during blood sampling, such as, for example, a meter that has a needle to sample blood on fingers, a strip to detect glucose in the blood sample, and a meter to read, display and store readings. One of such meters is OneTouch Ultra® 2 from LifeScan.

### SUMMARY OF THE INVENTION

One of embodiments of the present invention provides a device for noninvasive and continuous monitoring of blood glucose in a patient. The device comprises a unilateral permanent magnet configured to generate a static magnetic field, B₀, to a target area under the skin of the patient. The target area comprises blood vessels and tissue surrounding the blood vessels. The device also includes a transmitter (or transmit coil) configured to deliver a radiofrequency (RF) field, B₁, to the target area to generate magnetic resonance (MR) between the static magnetic field and proton nuclear spins in the target area, wherein at least a portion of the transmitter is positioned between the magnet and the skin. The device further comprises a sensor (or receiver coil) configured to detect a RF signal from the excited proton nuclear spins from the target area. Additionally, the device includes an electronic arrangement configured to process the detected RF signal (or signal data) from the sensor and generate a quantitative value corresponding to a level of blood glucose in a patient based on the signal data, more specifically, calculate the blood glucose level in the patient based on the signal data.

In another aspect of the invention, a method for monitoring of blood glucose level in a patient is provided. The method comprises a step for providing a static magnetic field to a target area under the skin of the patient, the target area comprising blood vessels and tissue surrounding the blood vessels. The method then delivers at least one pulse of a radiofrequency field to the target area to excite proton nuclear spins in the target area. The method also comprises a step for generating signal data corresponding to a RF signal detected by a sensor from the excited proton nuclear spins in the target area. The method then proceeds to analyze the signal data to generate a quantitative value corresponding to a level of blood glucose in a patient based on the data.

These and other aspects of the invention will become apparent to those skilled in the art after a reading of the following detailed description of the invention, including the figures and appended claims.

### BRIEF DESCRIPTION OF DRAWINGS

FIG. 1 shows a diagram for an exemplary device for non-invasive glucose monitoring of a patient, according to an exemplary embodiment of the present application.

FIG. 2 shows a block diagram flowchart for the exemplary device of FIG. 1.

FIG. 3 shows a circuit diagram for an exemplary embodiment of a transmit and receive subsystem for the exemplary device of FIG. 1

FIG. 4 shows a circuit diagram for an exemplary embodiment of a matching circuit for the transmit and receive subsystem of FIG. 3.

FIG. 5 shows a circuit diagram for an exemplary embodiment of a transmit/receive switch for the transmit and receive subsystem of FIG. 3.

FIG. 6 shows another exemplary device for non-invasive glucose monitoring of a patient, according to another exemplary embodiment of the present application.

FIG. 7 shows a block diagram of an exemplary embodiment of a connector for the exemplary device of FIG. 6.

FIG. 8 shows a block diagram of an exemplary embodiment for a transmit/receive circuit for the exemplary device of FIG. 6

FIG. 9 shows an exemplary method for non-invasive glucose monitoring of a patient, according to an exemplary embodiment of the present application.

FIG. 10 shows a diagram for a configuration the device of FIG. 1 and a depth and thickness of a target area to be excited and analyzed by the device.

FIG. 11 shows a diagram for a correlation between a RF pulse frequency with a depth and thickness of the target area for the configuration of FIG. 10.

FIG. 12 shows a diagram for a correlation between a RF pulse angle and phase of a pulse of the RF field from an RF source for the configuration of FIG. 10.

FIG. 13 shows an exemplary method for analyzing signal data to generate a quantitative value corresponding to a level of blood glucose in a patient, according to an exemplary embodiment of the present application.

FIG. 14 shows an exemplary embodiment for a set of 6 disk-shaped magnets suitable for use to impart a static magnetic field in the exemplary device of FIG. 1.

FIG. 15 shows a numerically-simulated distribution of the static magnetic field magnitude in a sagittal slice under one of the exemplary magnet of FIG. 14.

FIG. 16 shows data corresponding to measured static magnetic field magnitude along the vertical axis of one exemplary magnet of FIG. 14.

FIG. 17 shows an exemplary embodiment of a RF coil on a wrist model for use in the exemplary device of FIG. 6.

FIG. 18 shows data corresponding to distribution of RF field magnitude in an axial slice for the exemplary RF coil of FIG. 17.

FIG. 19 shows data corresponding to distribution of RF field magnitude in a sagittal slice for the exemplary RF coil of FIG. 17.

FIG. 20 distribution of RF field direction in a sagittal slice for the exemplary RF coil of FIG. 17.

FIG. 21 shows data comparing blood glucose levels measured by an exemplary embodiment of a device of the present application to blood plasma glucose levels measured using a conventional invasive, finger-pricking glucose meter in human study subjects.

FIG. 22 shows an example of RF transmit and receive signals from single acquisition data.

FIG. 23 shows a signal demodulated from the signals of FIG. 22.

FIG. 24 shows a Free Induction Decay (FID) signal of the demodulated signal of FIG. 23.

FIG. 25 shows a spectrum of the FID signal of FIG. 24.

### DETAILED DESCRIPTION

The present application provides a device and method for blood glucose monitoring that is non-invasive, and preferably painless, while providing quantitative measurements that are directly correlated to blood plasma glucose levels of a patient. In particular, the device and method of the present application utilize magnetic resonance spectroscopy (MRS) to generate signal data corresponding to a signal (e.g., a radiofrequency (RF) or magnetic resonance (MR) signal) from a region (e.g., a target area) under the skin of the patient and analyzing the signal data to generate a quantitative value corresponding to a level of blood glucose in the patient. The term “target area” as used herein refers to a region of tissue within a patient's body, which can be a three-dimensional region and is not limited to a two-dimensional area along a single plain. It is noted that the present application provides a non-invasive way, i.e., without the need for extracting a blood sample from a patient, to quantify blood glucose levels of the patient that directly correlates, and therefore, pinpoints the signal data to the blood glucose levels of the patient. This non-invasive monitoring of blood glucose may reduce the level of discomfort, such as pain, experience by patients as compared to invasive blood sampling for the purpose of blood glucose testing and/or monitoring. More particularly, this non-invasive monitoring is believed to be painless to the patient. Additionally, the device and method of the present application provides a more accurate way for measuring and quantifying blood glucose levels in the patient as compared to attempts at indirect measurements (e.g., measurements that are correlated to other physiological variable that are affected by, but not directly correlated to, changing levels of blood glucose).

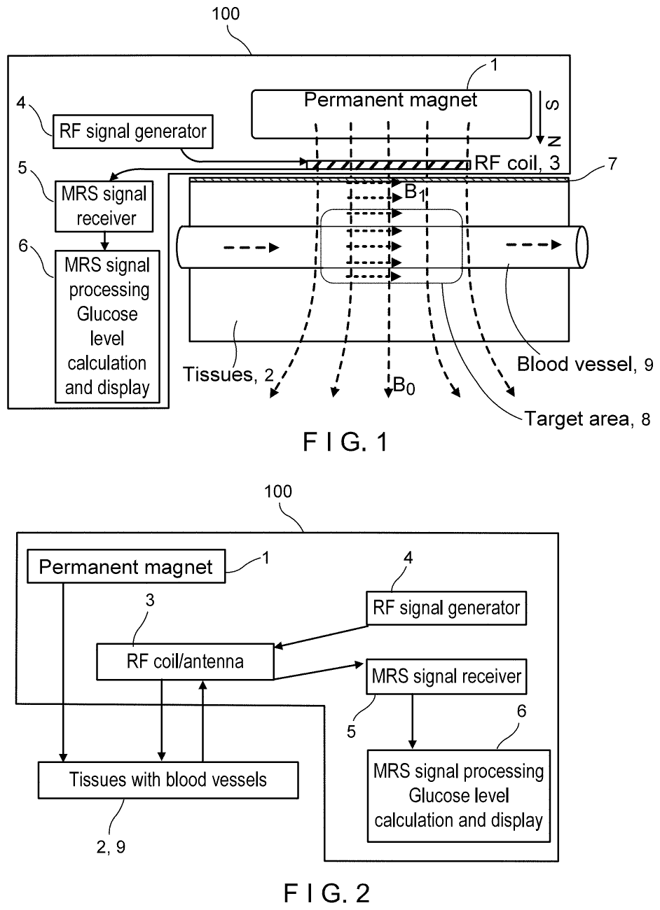

FIGS. 1 and 2 show an exemplary embodiment for a device 100 (e.g., blood glucose monitoring meter) for non-invasive glucose monitoring of a patient. Specifically, FIG. 1 shows an exemplary configuration of the device 100 when it is placed over the skin of the patient in an operating configuration. FIG. 2 shows a block diagram flowchart illustrating the exemplary device 100 of FIG. 1. The device 100 may be a small glucose meter that is significantly smaller in size than conventional magnetic resonance imaging (MRI) machines used in hospital settings. In particular, the device 100 may be a portable device that is suitably sized and shaped to be carried by the patient. The term “micromagnetic resonance spectroscopy” or “μMRS” as used herein refers to a portably sized MRS system or device that is suitably sized and shaped to be carried by the patient. More particularly, the device 100 may be suitably sized and shaped to be portable and wearable on a patient's body. For example, in one embodiment, the device 100 is suitably sized and shaped to be worn on or around a patient's wrist, arm, neck, or other body parts. Preferably, the device 100 is suitably sized and shaped so that it is easily portable and wearable by the patient throughout the day. For example, the device 100 may be suitably sized to have an average diameter of less than 2.0 inches, less than 1.5 inches, or less than 1.0 inches. As another example, the device 100 may be incorporated into wearable everyday items, such as, for example, a watch strap, armband, neck ring, etc. In one example, the device 100 is suitably sized and shaped to be worn on or around a patient's wrist, e.g., a watch-sized device, or a sensing device incorporated into a watch-sized device.

As shown in FIGS. 1 and 2, the device 100 comprises a magnet 1 for generating a static magnetic field, B₀, preferably, a unilateral static magnetic field. For example, the magnet 1 is a permanent magnet that generates a unilateral magnetic field. In particular, the magnet 1 is selected to generate a static magnetic field, B₀, having a magnetic field strength that is sufficient for penetrating past the skin 7 of the patient when the device 100 is in use (e.g., placed over the skin 7 of the patient in an operating configuration, or worn on the body of the patient). Specifically, the magnet 1 is selected to generate a static magnetic field, B₀, having a magnetic field strength sufficient for penetrating the skin 7 to reach underlying tissue 2. The underlying tissue 2 includes blood vessels 9 (e.g., arteries, veins and capillaries) extending throughout the tissue 2. More particularly, the static magnetic field, B₀, (which may, for example, be a unilateral magnetic field) provided by the magnet 1 is sufficiently strong so that when the device 100 is in use, the static magnetic field, B₀, penetrates the skin 7 and extends across blood vessels 9 under the skin 7. In particular, the magnet 1 (e.g., a unilateral magnet) is configured to generate a static magnetic field, B₀, having a magnetic field strength sufficient to reach a target area 8 under the skin 7 of the patient, and more particularly, impart the static magnetic field, B₀, throughout the target area 8. As shown in FIGS. 1 and 2, the target area 8 includes a portion of one or more of the blood vessels 9 as well as a portion of tissue 2 surrounding the blood vessels 9. It is contemplated that the magnet 1 can have a low strength, as compared to other magnets that are generally commercially available, while being sufficiently strong to impart the static magnetic field, B₀, throughout the target area 8. For example, the magnet 1 may have a strength from about 0.05 Tesla to about 0.5 Tesla, from about 0.05 Tesla to about 0.3 Tesla, from about 0.1 Tesla to about 0.3 Tesla, or from about 0.2 Tesla to about 0.3 Tesla. In one example, the magnet may have a strength of 0.234 Tesla.

The magnet 1 is suitably sized and shaped for incorporation into a wearable device. Specifically, the magnet 1 may be suitably sized and shaped for comfortable wearing by the patient on a part of the body, e.g., wrist, arm, neck, etc. For example, the magnet 1 may be suitably sized and shaped to be wearable on a wrist of the patient (e.g., having approximately the size of a quarter or a wristwatch face). In some embodiments, the magnet 1 has a disk shape having a circular or substantially circular cross-sectional shape and a thickness that is substantially smaller than its cross-sectional diameter. For example, the magnet 1 may have a disk shape having a diameter from about 0.5 inches to about 2.0 inches, or from about 1.0 inches to about 2.0 inches, and a thickness from about 1/16 inches to about ⅛ inches. In an exemplary embodiment, the magnet 1 is a disk-shaped magnet having the dimensions 1.26″×⅛″ (diameter×thickness). Furthermore, the magnet 1 in this embodiment is preferably suitably sized and shaped to be wearable and selected for providing a sufficiently strong static magnetic field, B₀, for penetrating the skin 7 and extending across blood vessels 9 under the skin 7 to align glucose molecules within the patient's blood stream to static magnetic field, B₀. In one example, the magnet 1 is a permanent magnet having a disk shape with the dimension of 1.26″×⅛″ (diameter×thickness) and a strength of 0.234 Tesla.

The device 100 further comprises a RF transceiver 2 for delivering a RF field and detecting a MR or RF signal. In particular, the RF transceiver 2 may comprise a transmit/receive coil (or antenna) for delivering a RF field, B₁, to excite proton (¹H) nuclear spins in the blood (including glucose molecules contained therein) and tissues of the target area 8 aligned by the B₀ field, and to receive signals generated by the excited spins. Although device 100 is described herein with respect to a transmit/receive coil, it is contemplated that a transmit/receive antenna can also be used and have similar characteristics as described herein for the transmit/receive coil. The RF transceiver 2 is suitably sized and shaped for incorporation into a wearable device. Specifically, the RF transceiver 2 may be suitably sized and shaped for comfortable wearing by the patient on a part of the body, e.g., wrist, arm, neck, etc. In one particular embodiment, the RF transceiver 2 may be suitably sized and shaped to wrap around a wrist of the patient (e.g., in the form of a bracelet or watch band surrounding the wrist). As shown in FIGS. 1 and 2, the RF transceiver 2 is operably connected to a signal generator 4 (e.g., a RF signal generator) and a signal receiver 5 (e.g., a MRS signal receiver). The signal generator 4 comprises any suitable component(s) (e.g., a transmit power amplifier) for powering the RF transceiver 2 to deliver a RF field in the manner discussed above. The signal receiver 5 comprises any suitable component(s) (e.g., a receive preamplifier) for detecting the MR or RF signal generated from excited proton nuclear spins caused by the RF field. Although FIGS. 1 and 2 illustrate an embodiment of the RF transceiver 2 as a single RF coil/antenna that can be used to both deliver a RF field and detect a MR or RF signal, it is contemplated that the RF transceiver 2 may comprise two distinct and separate components: a RF source (e.g., a RF transmitter), and a sensor (e.g., a receiver coil). In particular, the RF source is operably connected to the RF signal generator 4 and the sensor is operably connected to the MRS signal receiver 5. However, a single RF transceiver 2, as illustrated in FIGS. 1 and 2, may serve as both the RF source and the sensor. The RF source and the sensor are further discussed in detail below.

The RF source comprises any suitable RF transmitting component (e.g., a transceiver, a transmitter, a coil and/or an antenna) for generating a dynamic magnetic field (continuously or in pulses) at a suitable radiofrequency (e.g., a RF field) for excitation (e.g., excitation of proton (¹H) nuclear spins) in blood (including glucose molecules in the blood) circulating through blood vessels within the body of the patient. The RF transmitting component is suitably sized and shaped for incorporation into a wearable device. The RF transmitting component in the embodiment shown in FIGS. 1 and 2 is configured to deliver a RF field, B₁, to an area under the skin 7 of the patient that is also within the static magnetic field, B₀, field provided by the magnet 1. Specifically, the RF transmitting component is configured to deliver a RF field, B₁, having sufficient strength to penetrate the skin 7 and reach the target area 8 under the skin 7 when the device 100 is in use. As shown in FIG. 1, when the device 100 is in use, the RF transmitting component in this embodiment is positioned to deliver the RF field, B₁, to the target area 8, which includes a portion of the tissue 2, a portion of the blood vessels 9, and/or blood circulating through the target area 8 via the blood vessels 9. In particular, the RF transmitting component is configured to provide a RF field, B₁, at a suitable strength to penetrate the skin 7 and at a suitable radiofrequency to excite the underlying tissue 2. Specifically, the RF transmitting component is configured to deliver a RF field, B₁, to the target area 8 at a suitable radiofrequency for exciting proton (¹H) nuclear spins in the target area 8, including blood having glucose molecules therein circulating through the target area 8 via the blood vessels 9. As discussed above and shown in FIG. 1, the target area 8 is also subject to, and preferably aligned by, the static magnetic field, B₀, field provided by the magnet 1. Therefore, the RF transmitting component is arranged to deliver a RF field, B₁, that overlaps with the static magnetic field, B₀, field provided by the magnet 1 within at least the target area 8. In one embodiment, the RF transmitting component is arranged within the device 100 such that the RF field, B₁, is perpendicular or substantially perpendicular to the static magnetic field, B₀ provided by the magnet 1. For example, the RF transmitting component is arranged within the device 100 such that at least a portion of the RF transmitting component is positioned between the magnet 1 and the skin 7 when the device is in use.

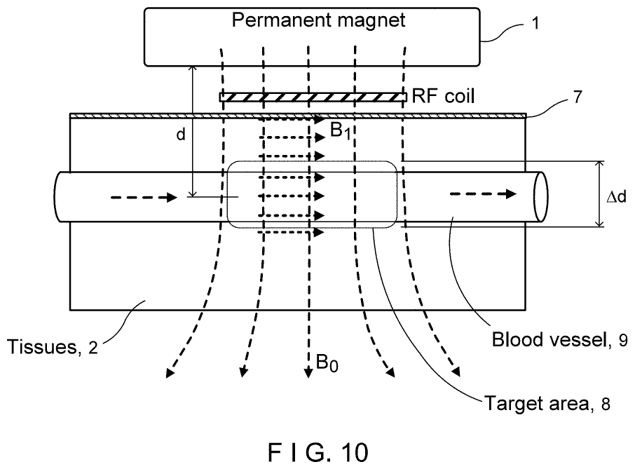

Furthermore, the RF transmitting component is preferably safe for use within close contact of a patient's skin. For example, the RF transmitting component is configured to operate only within frequencies that are safe for use adjacent to the skin and/or does not generate excessive external heat so as to cause discomfort or damage to the skin of the patient. For example, the RF transmitting component is configured to provide a dynamic magnetic field with in a radiofrequency range of about 0.425 MHz to about 42.5 MHz. In some embodiments, the RF transmitting component is configured to generate a pulse of a dynamic magnetic field having a radiofrequency range within a narrow bandwidth that is also suitable for exciting proton (¹H) nuclear spins in blood (including glucose molecules in the blood) circulating through blood vessels within the body of the patient and tissue surrounding the blood vessels. For example, the RF transmitting component is configured to generate a dynamic magnetic field having a radiofrequency range within a focused, narrow bandwidth ranging from 2.1 MHz to 4.2 MHz. Additional criteria for selecting the narrow bandwidth of radiofrequency range are discussed further below illustrated in FIGS. 10 through 12.

The sensor comprises any suitable component (e.g., a receiver coil) detecting a MR or RF signal. The sensor is suitably sized and shaped for incorporation into a wearable device. Specifically, the sensor is configured to detect a signal generated by the proton (¹H) nuclear spins excited by the RF field, B₁, provided by the RF transmitting component. In particular, the sensor is positioned within the device 100 so that when the device 100 is in use, the sensor generates signal data corresponding to a signal detected by the sensor from the proton (¹H) nuclear spins excited by the RF field, B₁ in the target area 8. The sensor is configured to generate signal data corresponding to signal detected from excited proton (¹H) nuclear spins from tissue 2 and blood vessels 9 within the target area 8, and, in particular, blood (including glucose molecules contained therein) circulating through the target area 8 via the blood vessels 9. For example, as shown in FIG. 1, the sensor is positioned within the device 100 in this embodiment such that at least a portion of the sensor is positioned between the magnet 1 and the skin 7 when the device is in use. The sensor may be operably connected to a processing arrangement (e.g., via the signal receiver) to provide (e.g., transmit) the signal data corresponding to the detected signal to the processing arrangement for analysis, as will be discussed further below. As discussed above, the sensor may be a separate component from the RF source or may be integrated with the RF source as part of the RF transceiver 2 in a single unit (e.g., a single RF coil/antenna). For example, the RF transceiver 2 may comprise a unitary coil formed from a single wire, multiple-turn wire, or a piece of conductive material. In particular, the unitary coil is a solenoid coil or a portion of a solenoid coil. The unitary coil may be formed from any suitable material for conducting a current therethrough (e.g., an electrical conductive material, a metal), preferably, copper.

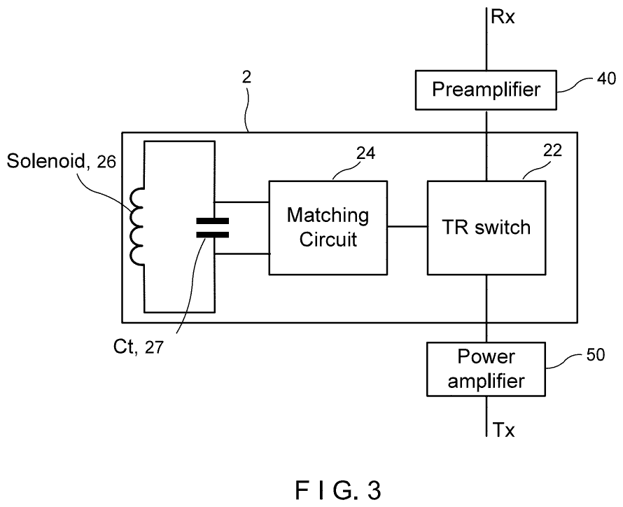

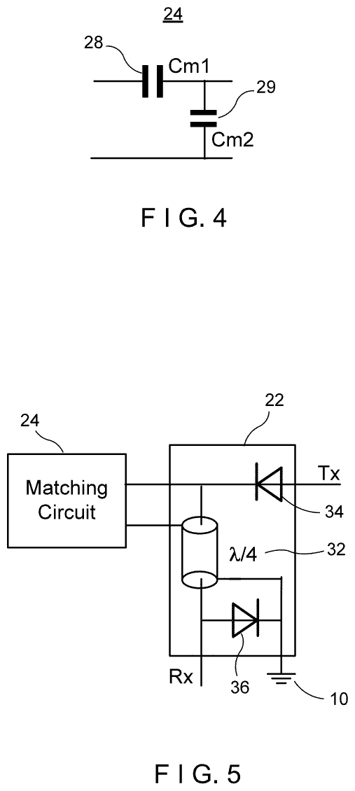

In some embodiments, the RF transceiver 2 is configured to reversibly switch, preferably automatically switch as directed by a processing arrangement, between a transmitting mode for delivering a RF field and a receiving mode for detecting a MR or RF signal. In particular, when the RF transceiver 2 is in the transmitting mode, it operates as the RF source, and when the RF transceiver 2 is in the receiving mode, it operates as the sensor, as discussed above. FIGS. 3 through 5 show an exemplary embodiment for a transmit and receive subsystem for the device 100, which includes the RF transceiver 2, the signal generator 4 (shown in FIG. 3 as comprising a power amplifier 50 operably connected to other transmission (Tx) components for delivering a RF field) and the signal receiver 5 (shown in FIG. 3 as comprising a preamplifier 40 operably connected to other reception (Rx) components for receiving a MR or RF signal). As shown in FIG. 3, the RF transceiver 2 comprises a solenoid coil 26. The solenoid coil 26 may be suitably sized and shaped for incorporating into a wearable device. Preferably, the solenoid coil 26 is suitably sized and shaped to surround a wrist of a patient, wherein a longitudinal axis through the solenoid coil 26 lies along a length of a patient's arm when the device is in use. The solenoid coil 26 may be made from any suitable materials (e.g., metal) for conducting a current therethrough. Preferably, the solenoid coil 26 is made from copper, and more specifically, copper braid. In this embodiment, the solenoid coil 26 is configured to be tuned to a desired working frequency and operably connected to a tuning capacitor (Cₜ) 22 and a matching circuit 24, as shown in FIG. 3. The working frequency of the solenoid coil 26 may be, for example, within the same ranges as described above for RF transmitting component. The matching circuit 24 comprises two additional tuning capacitors (Cₘ₁, Cₘ₂) 28, 29 arranged in the manner as shown in FIG. 4. In this embodiment, the flexibility and robustness of the solenoid coil 26 can be maximized by the tuning capacitor (Ct) 22 and the matching circuit 25, which can be placed on a small interface board. The RF transceiver 2 further comprises a transmit/receive switch 22. As shown in FIG. 3, the transmit/receive switch 22 is connected to the matching circuit 24 and configured to operate with the matching circuit 24 to switch the solenoid coil 26 between the transmitting mode and the receiving mode. An embodiment of the transmit/receive switch 22 is further shown in detail in FIG. 5. As shown in FIG. 5, the transmit/receive switch 22 comprises a first diode 34 operably connecting the matching circuit 24 to the signal transmitter 4 (e.g., transmission (Tx) components for delivering a RF field). The transmit/receive switch 22 also comprises a transmission cable 32 (e.g., a quarter-wave impedance transformer (λ/4)). The transmission cable 32 operably connects the matching circuit 24 to the second diode 36. The second diode is connected to the signal receiver 5 (e.g., reception (Rx) components for receiving a MR or RF signal) and a power source 10 (discussed further below), in the manner shown in FIG. 5. It is noted that the label Tx as used in FIG. 5 can include the preamplifier 40 as well as other reception components for receiving a MR or RF signal, which is different use of the label Tx in FIG. 3. Similarly, the label Rx as used in FIG. 5 can include the power amplifier 50 as well as other reception components for receiving a MR or RF signal, which is also different use of the label Rx in FIG. 3.

The device 100 further comprises a processing arrangement is configured to execute instructions stored on a computer accessible medium (e.g., memory storage device). The computer-accessible medium may, for example, be a non-transitory computer-accessible medium containing executable instructions therein. The processing arrangement is shown in FIGS. 1 and 2 in combination with an outputting arrangement, as part of a processing and display component 6 for MRS signal processing glucose level calculation and display. In one exemplary embodiment, the processing and display component 6 may comprise a micro-processor for controlling RF signal generation, transmission and reception, analysis of acquired signal data to separate glucose signal from other tissue signals and to calculate glucose concentration in the blood, and/or to display, store and manage readings. However, it is contemplated that the processing arrangement and the outputting arrangement can be separate, independent aspects of the processing and display component 6 and are not necessarily provided in combination in a single component. Although FIGS. 1 and 2 shows only a connection between the signal receiver 5 and the processing and display component 6, it is contemplated that the RF transceiver 2 (including the RF source and sensor as separate components or in combination), the signal generator 4, the signal receiver 5, and/or the outputting arrangement can be operably connected to the processing arrangement.

The processing arrangement may be configured to determine a level of blood glucose in the patient based on signal data corresponding to the detected RF signal from the sensor. In addition, the processing arrangement may also be configured to control RF signal generation by the RF source, receive data corresponding to the detected RF signal from the sensor, process the data corresponding to the detected RF signal from the sensor, and determine a level of blood glucose in the patient based on the signal data corresponding to the detected RF signal from the sensor.

The processing arrangement in this embodiment controls the RF transceiver 2 (or the RF source) and the RF signal generator 4 for providing the RF field, B₁, and receive and analyze signal data corresponding to signals detected by the RF transceiver 2 (or the sensor) from excited proton (¹H) nuclear spins from the target area 8. In particular, the processing arrangement analyzes the signal data to generate a quantitative value corresponding to a level of blood glucose in the patient based on the signal data. More particularly, the processing arrangement analyzes the signal data to determine a concentration of glucose in blood circulating through the target area 8 via the blood vessels 9 based on the signal data. In one embodiment, the processing arrangement extracts/separates blood glucose data from the signal data generated by tissues surrounding the blood vessels. The blood glucose data corresponds to a component of the detected signal contributed by glucose in the blood circulating through the target area. The blood glucose data is analyzed by the processing arrangement to determine glucose concentration in the blood plasma (similar to what is reported by clinical laboratory). The processing arrangement may be incorporated within the device 100 or may be part of a system comprising the device 100 and a separate device that is in communication with the device 100 via any suitable communications and/or logical connections. For example, the device 100 may further include radio antennas or any other suitable communications device for interfacing with an external processing arrangement, such as, for example, a computer or a smartphone.

The outputting arrangement is configured for outputting the results generated by the processing arrangement based on the analysis of the signal data. In particular, the outputting arrangement comprises a user interface for displaying a quantitative value (e.g., concentration) corresponding to a level of blood glucose in the patient based on the signal data, e.g., a display or a smartphone. The outputting arrangement may be incorporated within the device 100 or may be part of a system comprising the device 100 and a separate device, e.g., a computer or a smart phone, in communication with the device 100 via any suitable communications and/or logical connections.

The device 100 further comprises a power source 10 providing power to control and operate the device 100. In particular, the power source 10 is operably connected to the RF source, the sensor, the signal generator 4, the signal receiver 5, the processing arrangement and/or the outputting arrangement to provide power to control and operate the device 100. In some exemplary embodiments, the RF source may comprise a RF transmitting coil and/or antenna operably connected to the power source 10. Those skilled in the art will understand that various known suitable sources of power may be used. For example, the power source may comprise a battery or a connection to an external source of power. In particular, the power source may comprise a rechargeable battery device. The battery may be suitably sized and shaped to fit within a wearable device while providing sufficient power to the device 100 to control and operate the device 100 for monitoring glucose levels of a patient, and more particularly, continuously monitoring glucose levels throughout the day, so that patient can wear the device throughout the day without need to recharge the battery. For example, the battery may be configured to provide sufficient power for continuous operation of the device 100 at least, e.g., during day time, 8 hours, 12 hours, 1 day, etc.

The device 100 or a system comprising the device 100 may also include an input device, such as a touchable screen or button, or an interface via a computing device, that permits manual triggering of a blood glucose test. The input device may be part of the device 100 or a separate device in communication with the device 100 via any suitable communications and/or logical connections.

In one exemplary embodiment, the device 100 comprises: (1) a wristwatch-sized permanent magnet at ˜0.234 Tesla, (2) a transmit/receive RF coil/antenna and associated power supply electronics, and (3) an integrated electronic for glucose quantification, display and wireless transmission to accessories such as smart phones.

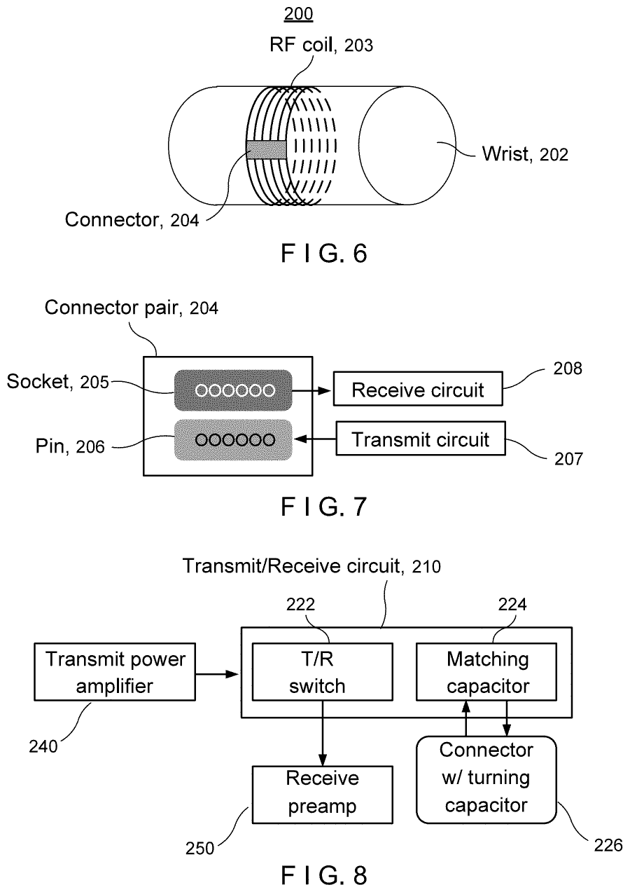

FIGS. 6 through 8 show another exemplary embodiment for a device 200 (e.g., blood glucose monitoring meter) for non-invasive glucose monitoring of a patient. The device 200 shown in FIGS. 6 and 8 is substantially similar to the device 100 described above, except in the portions further described below. The device 200 comprises a RF transceiver 203 comprising a coil or an antenna sized and shaped to surround a wrist 202, or a portion of a wrist 202, of a patient. In particular, the RF transceiver 203 comprises a coil or an antenna sized and shaped to surround the wrist 202, wherein a longitudinal axis through the coil/antenna lies along a length of a patient's arm. In some embodiments, the coil/antenna may comprise a unitary coil formed from a single wire, multiple-turn wire, or a piece of conductive material. In particular, the unitary coil is a solenoid coil or a portion of a solenoid coil. The coil/antenna may be formed from any suitable material for conducting a current therethrough (e.g., an electrical conductive material, a metal), preferably, copper. The coil/antenna may be in the form of a bracelet or watch band surrounding the wrist 202. In some embodiments, the coil/antenna may include any suitable connector 204 having an open/close clasp to allow for opening and closing of the coil. For example, the coil/antenna may comprise N-turns of copper wires configured to configured to be wrapped around a wrist of a patient with a connector 204 having an open/close clasp to which a tuning capacitor may be attached (a combination of the connector 204 and the tuning capacitor is labelled as 226 in FIG. 8).

The connector 204, as shown in FIG. 7, comprises an array of sockets 205 and an array of pins 206 configured to reversibly engage and disengage the array of sockets. The connector 204 is configured to be reversibly movable between an open configuration and a closed configuration such that the coil can be easily worn and removed from the wrist 202 of a patient. In particular, the coil/antenna and connector 204 is in an open configuration when the array of sockets 205 are separated from the array of pins 206 to create a longitudinal opening along a side of the coil/antenna to allow for the wrist 202 of the patient to slide therethrough for wearing and removal of the device 200 from the wrist of the patient. The coil/antenna and connector 204 are in a closed configuration when the array of sockets 205 are engaged with the array of pins 206 to re-connect the coil across the longitudinal opening formed in the open configuration. In particular, when the coil/antenna and connector 204 are in the closed configuration, ends of the coil/antenna across the longitudinal opening, which define a break between portions of the coil/antenna at the location of the connector 204, are re-connected to resume a shape corresponding to that formed from a single wire, e.g., where each turn of the coil/antenna is electrically reconnected to permit a current therethrough while the windings in each turn of the coil/antenna are insulated from each along the sides of the wire, in particular, in the shape of a solenoid coil.

The device 200 further comprises a magnet, similar to the magnet 1 for generating a static magnetic field, B₀, as discussed above for device 100. In particular, the magnet of device 200 may be a unilateral permanent magnet that is sized and shaped to be wearable on the wrist. In one particular embodiment, the magnet of device 200 may have a disk shape approximately having the size of a quarter or a wristwatch face. The magnet is tangentially attached to an external side of the coil. A portion of the coil/antenna may be positioned between the magnet and the skin of the patient when the device 200 is worn around the wrist 202 of the patient. The combination of the magnet and coil/antenna may have a size and shape similar to that of a watch. For example, the coil/antenna may have a circumference from about 130 mm to 210 mm, or from about 165 mm to about 197 mm.

As shown in FIG. 7, the array of pins 206 is operably connected to a transmit circuit 207 and the array of sockets 205 is operably connected to a receive circuit 208. The transmit circuit 207 comprises a plurality of electrical components operably connecting the array of pins 206 with a signal generator 240 (e.g., a RF signal generator), which can comprise any suitable component(s) (e.g., a transmit power amplifier) for powering the RF transceiver 203 to deliver a RF field. The receive circuit 208 comprises a plurality of electrical components operably connecting the array of sockets 205 with a signal receiver 250 (e.g., a MRS signal receiver), which can comprise any suitable components (e.g., a receive preamplifier) for detecting the MR or RF signal generated from excited proton nuclear spins caused by the RF field.

In one exemplary embodiment, the transmit circuit 207 and the receive circuit 208 may be integrated together as part of a transmit/receive circuit 210, as shown in FIG. 8. The transmit/receive circuit 210 comprises a transmit/receive switch 222 that is operably connected to a matching capacitor 224, which is operably connected to the connector 204 and tuning capacitor (collectively 226). The transmit/receive circuit 210 is configured to reversibly switch, preferably automatically switch as directed by a processing arrangement, between a transmitting mode for delivering a RF field and a receiving mode for detecting a MR or RF signal. In particular, the transmit/receive switch 222 is operably connected to the processing arrangement to automatically direct the transmit/receive switch 222 to switch between the transmitting mode and the receiving mode, as directed by the processing arrangement without further manual input from the patient.

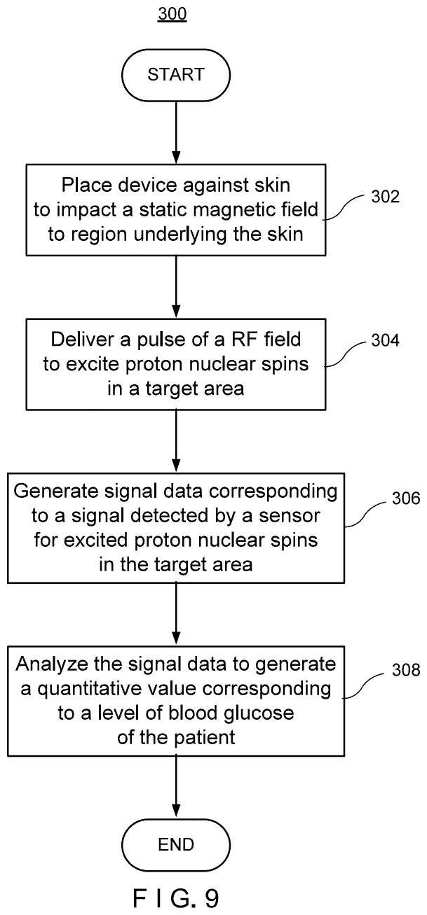

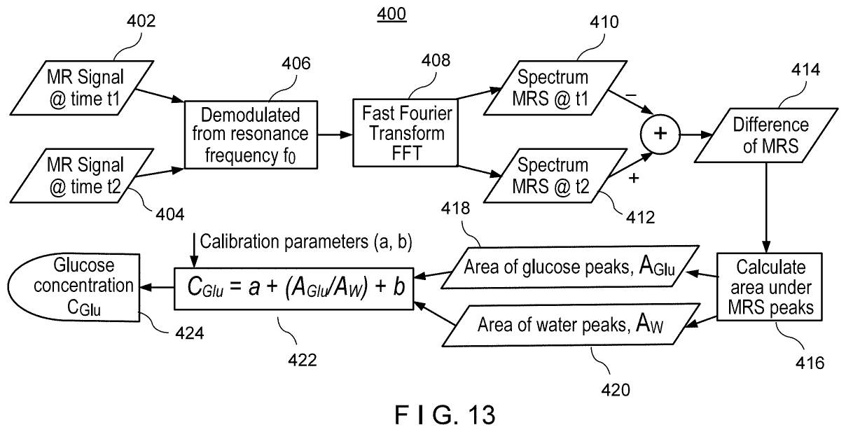

The present application also includes a method for non-invasive glucose monitoring of a patient. In particular, the method provides a non-invasive method for in-vivo or in-situ monitoring of blood glucose levels in a patient. An exemplary method 300 is shown in FIG. 9. In step 302, the user may initiate use of a non-invasive glucose monitoring device or meter of the present application, by placing the device over an area of skin to impart a static magnetic field, B₀, to a region under the skin, as discussed above. In one example, a magnet (e.g., a unilateral permanent magnet) is used to provide the static magnetic field, B₀, to the region under the skin, which can include blood vessels, blood circulating through the blood vessel, and surrounding tissue. As indicated in step 304, a RF source delivers a RF field, B₁, to a target area under the skin of the patient that is also within the static magnetic field, B₀, field provided by the magnet. For example, the RF source delivers a pulse of the RF field, B₁, at an appropriate frequency such the RF field, B₁, extends beyond the surface of the skin and imparts the RF field, B₁, to the target area. The target area includes tissue and blood vessels under the skin, including glucose present within the patient's blood stream. The RF field, B₁, is delivered to the target area as described above and further discussed below and illustrated in FIGS. 10 through 12 for adjusting operating parameters of the RF sources to select for a desired target area. The RF field, B₁, excites proton (¹H) nuclear spins in the target area, which can include tissue 2, blood vessels 9, and blood circulating through the region via the blood vessels containing glucose molecules therein. In step 306, a sensor detects a signal from the proton (¹H) nuclear spins excited by the RF field, B₁ in the target area, and generates signal data based on the detected signal. The sensor may detect a signal at single time, at multiple times, or over a period of times. In step 308, a processing arrangement receives and analyzes the signal data corresponding to a signal detected by the sensor from the proton (¹H) nuclear spins excited by the RF field, B₁ in the target area. The processing arrangement may first extract/separate blood glucose data from the signal data. The blood glucose data corresponds to a component of the detected signal contributed by glucose from blood circulating through the target area. The blood glucose data is further analyzed by the processing arrangement to generate a quantitative value (e.g., concentration) corresponding to a level of blood glucose in the patient. One exemplary method 400 for analyzing the signal data to generate a quantitative value corresponding to a level of blood glucose in the patient is shown in FIG. 13 and explained in further detail below. In some embodiments, the results from step 308 may be displayed on a user interface.

The method 300 may be repeated at any desired rate to repeatedly measure the patient's blood glucose levels. For example, the method 300 may be repeated to determine the patient's blood glucose level ad hoc (such as when manually directed by a user via an input through a user interface), based on a predetermined time schedule, or may continuously monitor the patient's blood glucose level throughout a period of time. The system may continuously monitor the patient's blood glucose level within any predetermine time frame, e.g., during day time, 8 hours, 12 hours, 1 day, etc. Each test may be conducted within a short period of time, such as, for example, within 5 mins, within 3 mins, within 60 seconds, or within 30 seconds. Preferably, the test may be conducted in real-time, or substantially in real-time, i.e., wherein the delay is not easily noticeable to a human, such as, for example, within 0.5 second, more preferably within 0.25 second.

In some embodiments, the static magnetic field, B₀, provided by a wearable-size magnet may generate an inhomogeneous field. However, the challenge of inhomogeneous B₀, field from the permanent magnet may be minimized by carefully selecting a desired target area under the skin that is imparted with a locally uniform B₀ field by the magnet in step 302, as further described below and illustrated in FIGS. 10 through 12. It is noted that this embodiment is different from the whole-body clinical MRI systems where spatial localization is achieved through the use of gradient subsystems, the embodiment shown in FIGS. 10 through 12 does not rely on a gradient subsystem and does not require the static magnetic field, B₀, and the RF field, B₁, to be uniform throughout. Instead, this embodiment may fine-tune the positioning and size of a desired target area by manipulating the frequency and bandwidth of a pulse of the RF field, B₁ delivered from the RF source, its flip angle (θ) and/or phase (ω). For example, this desired target area may be selected by adjusting operating parameters of the RF sources to select for a desired target area. In particular, selection of this desired target area may be achieved by adjusting the RF source to generate the RF field, B₁, at a frequency within a narrow bandwidth range (f₀±Δf) and at phase-varying composite flip angles during excitation (e.g., in step 304). More particularly, step 304 may include additional step for adjusting various operating parameters for a pulse of the RF field, B₁, delivered by the RF source, e.g., the RF pulse frequency, bandwidth (Δf), flip angle (θ) and phase (Φ) for excitation proton (¹H) nuclear spins in the desired target area and to generate signals for detection in step 304 from the desired target area.

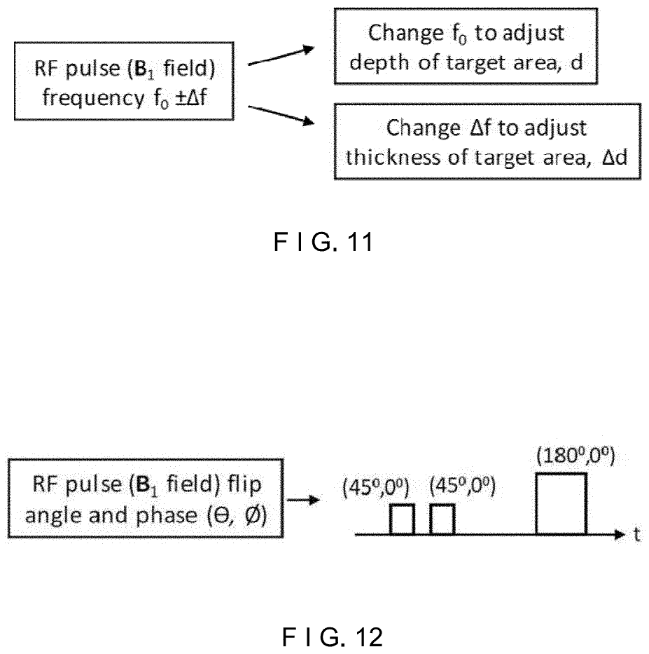

FIG. 10 shows the exemplary configuration of FIG. 1 where the depth (d) of the target area 8 is shown as a distance between the magnet 1 and a midway point across a thickness (Δd) of the target area 8. The target area 8, shown in FIG. 10, to which the RF field, B₁, is delivered is selected based on the configuration between the static magnetic field, B₀, and the RF field, B₁ (shown in FIG. 10 as overlapping and perpendicular or substantially perpendicular to one another), and as well as various operating parameters for a pulse of the RF field, B₁, delivered by the RF source, e.g., the RF pulse frequency, bandwidth (Δf), flip angle (θ) and phase (Φ) for excitation proton (¹H) nuclear spins in the desired target area. FIG. 11 shows a correlation between a RF pulse frequency with a depth and thickness of the desired target area. As shown in FIG. 11, an adjustment to an average of RF frequency (f₀) within a select range is used to adjust a depth (d) of the target area, and an adjustment to a span of the bandwidth range (Δf) is used to adjust a thickness (Δd) of the target area. The average RF frequency (f₀) and span of the bandwidth range (Δf) may be selected by the processing arrangement to direct the RF source to deliver a pulse of the RF field, B₁, to any desired target area under the skin 7 of the patient. More particularly, the positioning and size of the desired target area is selected such that a depth (d) of the target area 8 is sufficiently under the skin 7 to detect glucose levels in blood vessels and a thickness (Δd) of the target area 8 is sufficient to encompass capillaries and/or other blood vessels and produce detectable differences in a RF signal from proton (¹H) nuclear spins in the blood and tissues in the target area 8. FIG. 12 shows a correlation between a RF pulse angle (θ) and phase (Φ) of a pulse of the RF field, B₁, from the RF source. Specifically, the pulse of the RF field, B₁, delivered by the RF source, can be adjusted to provide a series of multiple flip angles, such as the angles shown in FIG. 12. As shown in FIG. 12, an adjustment to a RF pulse angle (θ) and phase (Φ) of a pulse of the RF field, B₁, from the RF source further adjusts the depth (d) and thickness (Δd) of the target area, and thereby fine-tuning the selection of the desired target area 8 for delivery of a pulse of the RF field, B₁, by the RF source.

FIG. 13 shows an exemplary method 400 for analyzing signal data corresponding to a signal detected by the sensor from the proton (¹H) nuclear spins excited by the RF field, B₁ in the target area 8 to generate a quantitative value (e.g., concentration) corresponding to a level of blood glucose in the patient. It is believed that the signal data can be analyzed to obtain quantitative measurements for blood glucose because glucose exhibits a unique chemical shift in magnetic resonance spectroscopy (e.g., a range of 3.2-3.9 ppm, or a range of 3.2-3.8 ppm) in magnetic resonance spectroscopy, distinct from circulating water (e.g., at 4.7 ppm or 4.8 ppm) in the vascular systems. Firstly, the difference in chemical shift between glucose and circulating water is large enough to separate themselves. For example, it may correspond to a frequency difference of 8.0-15.0 Hz or 10.0-16.0 Hz at 0.234 Tesla and may be detectable using a high-resolution (Δf=0.5 Hz) spectroscopy.

The processing arrangement receives from step 306 a first set of signal data 402 corresponding to a signal detected by the sensor for excited proton nuclear spins in the target area 8 at a first time point (t₁) and a second set of signal data 404 corresponding to a signal detected by the sensor for excited proton nuclear spins in the target area 8 at a second time point (t₂). In step 406, the processing arrangement demodulates the first and second sets 402, 404 of signal data from a resonance frequency of the device 100 and applies a Fast Fourier Transform (step 408) to the demodulated first and second set of signal data to obtain first and second sets 410, 412 of data corresponding to spectrum MRS at the first and second time points, respectively. In step 414, the first and second sets 410, 412 of data corresponding to spectrum MRS are subtracted to extract an MR spectrum corresponding to a component of the first and second set of signal data 402, 404 contributed solely by blood circulating through blood vessels within the target area 8. In particular, the processing arrangement removes contributions to the MRS signals from surrounding static tissues within the target area 8. Furthermore, the processing arrangement may also remove contributions to the MRS signals from fat at 3.4-3.5 ppm in magnetic resonance spectroscopy that overlaps on glucose peaks. In step 416, the processing arrangement determines an area 418 under the MR spectrum corresponding to glucose (e.g., portion of the MR spectrum at 3.2-3.9 ppm) and an area 420 under the MR spectrum corresponding to water (e.g., portion of the MR spectrum at 4.7 ppm). In step 422, the processing arrangement determines a quantitative value, in particular, a concentration value, corresponding to a level of blood plasma glucose in the patient using a ratio of the area 418 under the MR spectrum corresponding to glucose to the area 420 under the MR spectrum corresponding to water. Specifically, the processing arrangement determines an absolute glucose concentration in blood plasma, C_{Glu}, via a pre-set calibration using the following equation:

C_{Glu}=a*(A_{Glu}/A_{W})b

where a and b are pre-set calibration parameters. In some embodiments, the calibration parameters are determined in an one-time process for all devices utilizing exemplary method 400. The pre-set calibration parameters, a and b, may be pre-determined values stored within the device 100 or may be obtained prior to use of the device 100. For example, the pre-set calibration parameters, a and b, may be determined in an initial set up process and stored within the device for determining blood glucose concentrations based on measured MR spectra. In some embodiments, the pre-set calibration parameters may be re-determined after use of the device for a pre-determined period of time or may be manually triggered by a user. The method 400 described herein may be repeated N (e.g., 1-500) times to increase signal-to-noise ratio (SNR).

The device, system and/or method described in the present application advances daily glucose monitoring to a noninvasive (painless) and continuous way for diabetic patients and those who are living with a high level of blood glucose to use at home and/or outside. It is believed that the system and device of the present application provides a non-invasive way to directly measure blood glucose levels of a patient having comparable accuracy as those direct measurements obtained using invasive meters. The micro magnetic resonance spectroscopy (μMRS) device, system and/or method of the present application is designed to non-invasively and continuously monitor glucose level in the blood. It is contemplated that the μMRS device, system and/or method described herein may be configured to have the following properties:
Completely noninvasive (no need for blood sampling and painless),
Wearable on the wrist, arm, neck, or other body parts,
Able to provide a test result in <60 seconds,
Able to monitor glucose level continuously,
Having the potential to meet ISO 15197 (standards for glucose measurements),
Able to integrate with everyday items (watch strap, armband, and neck ring), and
Able to interface with smartphones.

The device, system and/or method described herein may provide sufficient accuracy so as to meet any suitable standards for glucose measurements. In one embodiment, the embodiments described in the present application may be suitable for meeting the validation standards set forth in ISO 15197:2013 standard (available at https://www.iso.org/standard/54976.html, which is incorporated by reference herein). More particularly, the embodiments of the present application may be sufficiently accurate that at least 95% of measurements obtained by the glucose meter described herein fall within ±15 mg/dL or ±15% of the laboratory reference result at blood glucose concentrations of <100 mg/dL and ≥100 mg/dL, respectively.

Those skilled in the art will understand that the exemplary embodiments of the processing arrangement described herein may be implemented in any number of manners, including as a separate software module, as a combination of hardware and software, etc. For example, the exemplary analysis methods may be embodiment in one or more programs stored in a non-transitory storage medium and containing lines of code that, when compiled, may be executed by at least one of the plurality of processor cores or a separate processor. In some embodiments, a system comprising a plurality of processor cores and a set of instructions executing on the plurality of processor cores may be provided. The set of instructions may be operable to perform the exemplary methods discussed herein. The at least one of the plurality of processor cores or a separate processor may be incorporated in or may communicate with any suitable wearable electronic device, including, for example, a mobile computing device, a smart phone, a computing tablet, a computing device, etc.

Such processing arrangement may be, e.g., entirely or a part of, or include, but not limited to, a computer/processor that can include, e.g., one or more microprocessors, and use instructions stored on a computer-accessible medium (e.g., RAM, ROM, hard drive, or other storage device). A computer-accessible medium (e.g., as described herein, a storage device such as a hard disk, floppy disk, memory stick, CD-ROM, RAM, ROM, etc., or a collection thereof) can be provided (e.g., in communication with the processing arrangement). The computer-accessible medium may be a non-transitory computer-accessible medium. The computer-accessible medium can contain executable instructions thereon. In addition or alternatively, a storage arrangement can be provided separately from the computer-accessible medium, which can provide the instructions to the processing arrangement so as to configure the processing arrangement to execute certain exemplary procedures, processes and methods, as described herein, for example.

Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

### EXAMPLES

### Example I

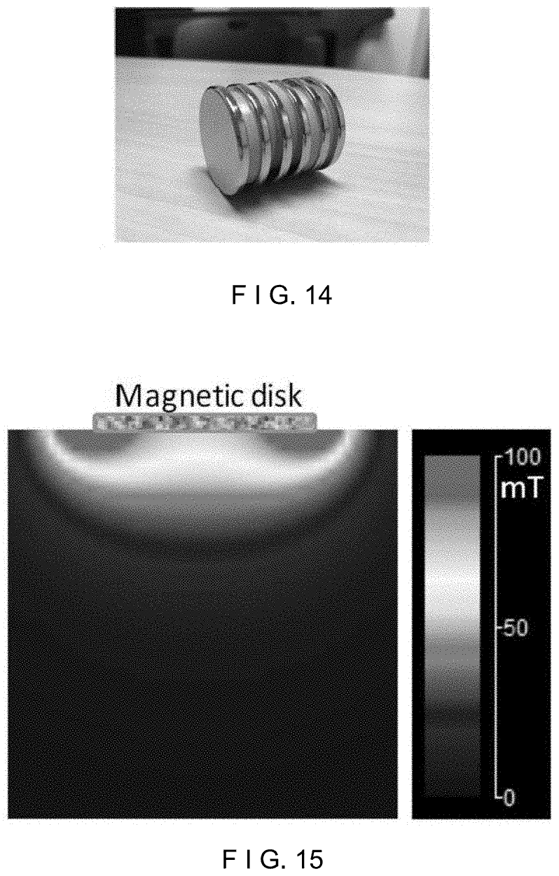

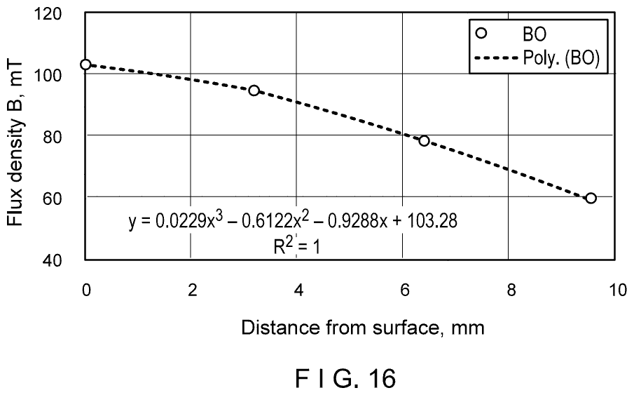

FIG. 14 shows an exemplary embodiment for a magnet 1 that is suitable for use in the device 100 described above. Specifically, FIG. 14 shows an exemplary set of 6 magnetic disks (D32×H1.6 mm²). FIG. 15 shows a numerically simulated static magnetic fields, B₀, generated by one of the exemplary magnetic disks of FIG. 14. Specifically, FIG. 15 shows a numerically-simulated distribution of the B₀ field magnitude in a sagittal slice under one of the exemplary magnetic disk, showing investigation depth. FIG. 16 shows measured B₀ field magnitude (by Gauss meter) along the vertical axis of one of the exemplary magnetic disks of FIG. 14, showing an investigation depth as large as 6.0 mm.

### Example II

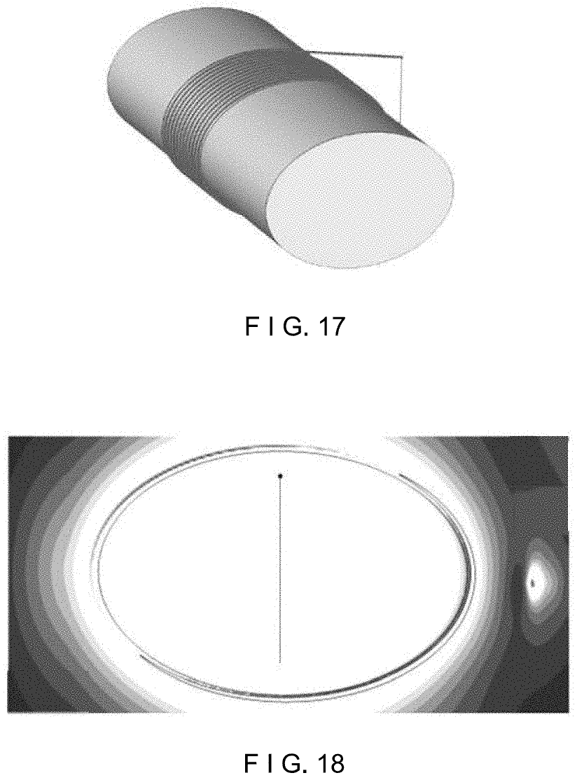

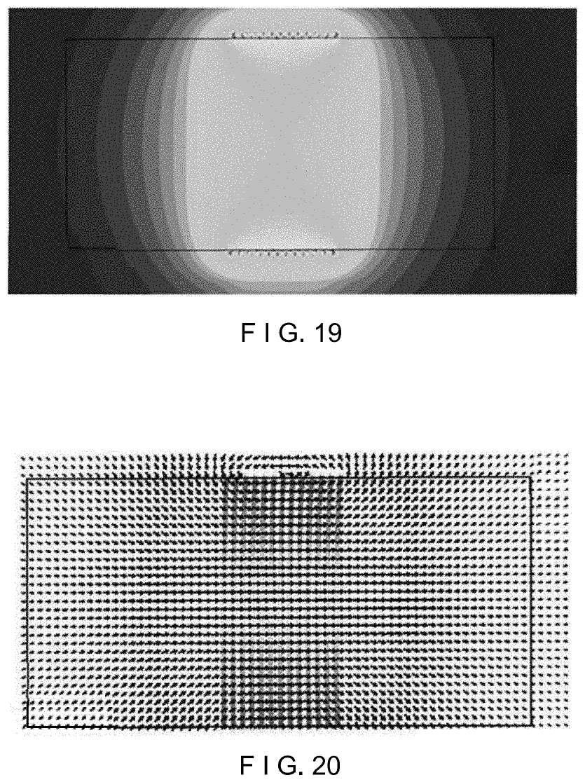

FIGS. 17 through 20 shows an exemplary embodiment of a RF coil on a wrist model that is suitable for use in the device 200 described above. Specifically, FIG. 17 shows a RF coil on a wrist model having the dimensions L26.2×H24.5×W37.0 mm³. FIGS. 18 through 20 show simulated corresponding B₁ radiofrequency field magnitude distributions for a device 200 having the RF coil of FIG. 17. In particular, FIG. 18 shows distribution of the B₁ field magnitude in an axial slice, with a black dot at the depth of 5.2 mm from the surface. FIG. 19 shows distribution of the B₁ field magnitude in a sagittal slice, showing investigation depth. FIG. 20 shows distribution of the B₁ field direction in a sagittal slice, showing a horizontal direction in the investigation area.

### Example III

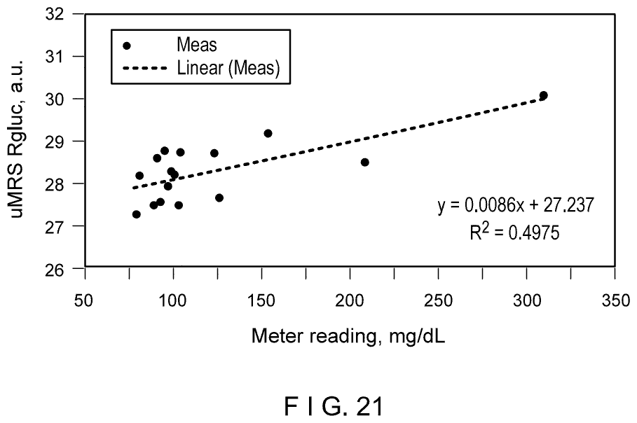

FIG. 21 shows data obtained from a subject study on seven Type II diabetes and nine healthy controls using an exemplary embodiment of device 100 (labeled as μMRS measurements) and a conventional invasive, finger-pricking glucose meter (plasma-calibrated). As can be seen in FIG. 21, the data shows a strong linear correlation between the μMRS measurements and the conventional invasive meter readings.

### Example IV

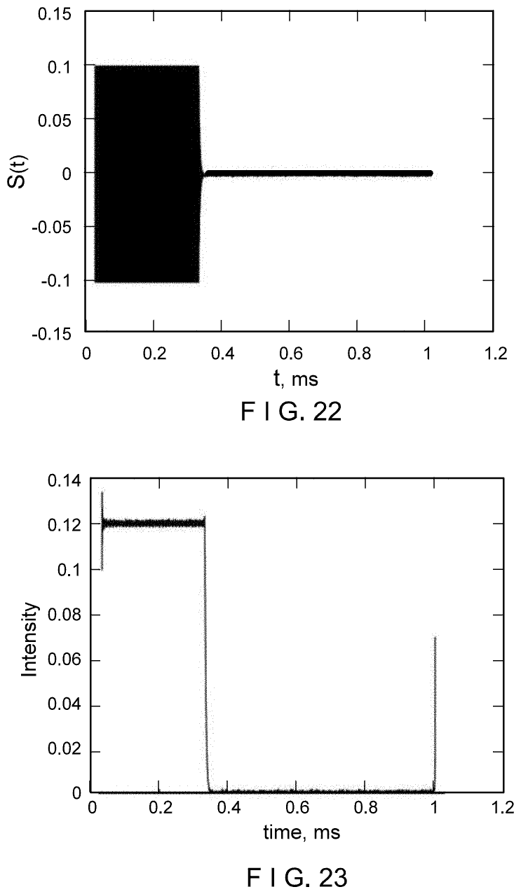

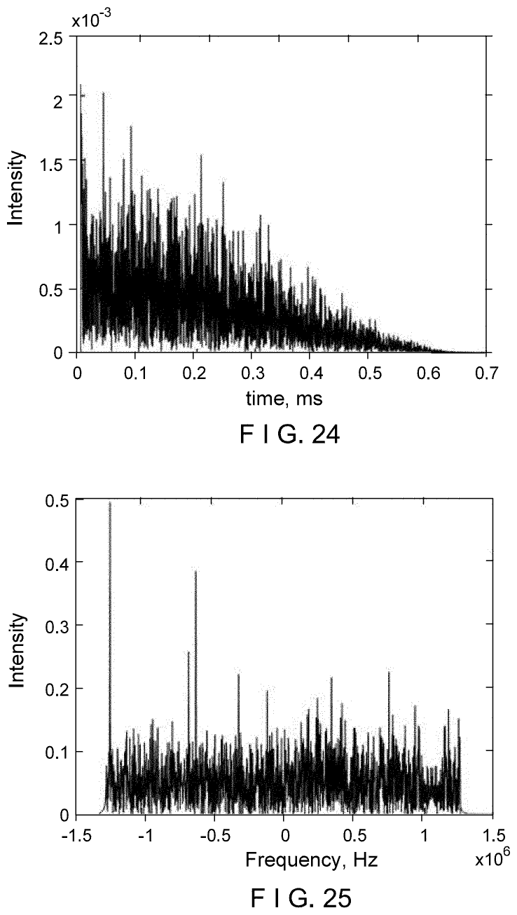

FIGS. 22 through 25 show an example of μMRS signals from single acquisition data. Specifically, FIG. 22 shows an example of RF transmit and receive signals. FIG. 23 shows a signal demodulated from the signals shown in FIG. 22 obtained in the same manner as step 406 described above. FIG. 24 shows a Free Induction Decay (FID) signal of the demodulated signal of FIG. 23. The FID was multiplied by a Harming windowing function for Fourier transformation used in the next step. FIG. 25 shows a Fourier spectrum of the FID signal of FIG. 24.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. A device for monitoring of blood glucose level in a patient, comprising:
a magnet configured to generate a static magnetic field to a target area under the skin of the patient, the target area comprising blood vessels and tissue surrounding the blood vessels;
a transmitter configured to deliver a radiofrequency (RF) field to the target area to excite proton nuclear spins in the target area, wherein at least a portion of the transmitter is positioned between the magnet and the skin;
a sensor configured to detect a RF signal from the excited proton nuclear spins in the target area; and
a processing arrangement configured to receive signal data corresponding to the detected RF signal from the sensor, and to generate a quantitative value corresponding to a level of blood glucose in a patient based on the data.

2. The device of claim 1, wherein the device is configured to be wearable on a body of the patient.

3. The device of claim 1, wherein the quantitative value corresponds to a concentration of glucose in blood circulating via the blood vessels through the target area.

4. The device of claim 1, wherein the magnet is a unilateral magnet.

5. The device of claim 1, wherein the transmitter and the sensor are separate components within the device.

6. The device of claim 1, wherein the transmitter and the sensor are integrated as part of a transceiver configured to reversibly operate in a transmitting mode for delivering the RF field and a receiving mode for detecting the RF signal.

7. The device of claim 6, wherein the transceiver comprises a switch movable between a first setting in which the switch controls operation of the transceiver in the transmitting mode and a second setting in which the switch controls operation of the transceiver in the receiving mode.

8. The device of claim 6, wherein the transceiver comprises a coil or an antenna.

9. The device of claim 8, wherein the coil or the antenna is formed from an electrical conductive material.

10. The device of claim 9, wherein the electrical conductive material is copper.

11. The device of claim 6, wherein the transceiver comprises a unitary coil formed from a single wire, a multiple-turn wire, or a piece of conductive material.

12. The device of claim 6, wherein the transceiver comprises at least a portion of a solenoid coil.

13. The device of claim 6, wherein the transceiver is configured to surround at least a portion a wrist of the patient.

14. The device of claim 8, wherein the transceiver comprises a connector configured to reversibly open and close the coil or the antenna.

15. The device of claim 14, wherein the connector comprises an array of sockets and an array of pins configured to reversibly engage and disengage the array of sockets.

16. The device of claim 15, wherein the connector is movable between an open configuration wherein the array of sockets are separated from the array of pins to create a longitudinal opening along a side of the coil or the antenna, and a closed configuration wherein the array of sockets are engaged with the array of pins to re-connect the coil or the antenna across the longitudinal opening formed in the open configuration.

17. A method for monitoring of blood glucose level in a patient, comprising:
providing a static magnetic field to a target area under the skin of the patient, the target area comprising blood vessels and tissue surrounding the blood vessels;
delivering at least one pulse of a radiofrequency field to the target area to excite proton nuclear spins in the target area;
generating signal data corresponding to a RF signal detected by a sensor from the excited proton nuclear spins in the target area; and
analyzing the signal data to generate a quantitative value corresponding to a level of blood glucose in a patient based on the data.

18. The method of claim 17, wherein the quantitative value corresponds to a concentration of glucose in blood circulating via the blood vessels through the target area.

19. The method of claim 17, wherein the static magnetic field is substantially perpendicular to the radiofrequency field.

20. The method of claim 17, wherein the analyzing step comprises extracting blood glucose data from the signal data, the blood glucose data corresponding to a component of the RF signal contributed by glucose in blood circulating via the blood vessels through the target area, and analyzing the blood glucose data to determine the quantitative value corresponding to the level of blood glucose in the patient based on the blood glucose data.
